(19) United States
(12) Patent Application Publication
(10) Pub. No.: US 2005/0215644 A1
(43) Pub. Date: Sep. 29, 2005

(54) Methods for increasing neurotransmitter levels using hydroxycitric acid

(75) Inventors: Debasis Bagchi, Concord, CA (US); Sunny E. Ohia, Pearland, TX (US); Harry G. Preuss, Fairfax Station, VA (US)

Correspondence Address: FLIESLER MEYER, LLP, FOUR EMBARCADERO CENTER, SUITE 400, SAN FRANCISCO, CA, 94111, US

(73) Assignee: InterHealth Nutraceuticals, Inc., Benicia, CA (US)

(21) Appl. No.: 11/081,176

(22) Filed: Mar. 16, 2005

Related U.S. Application Data
(60) Provisional application No. 60/554,653, filed on Mar. 19, 2004.

(51) Int. Cl. A61K 31/19

(52) U.S. Cl. 514/574

(57) ABSTRACT

Composition and methods for increasing neurotransmitter levels of dopamine and serotonin in a subject through administration of (−)-hydroxycitric acid.

### PRIORITY CLAIM

This application claims priority to U.S. Provisional Patent Application Ser. No: 60/554,653, titled: Methods for Increasing Neurotransmitter Levels, inventors: Debasis Bagchi and Sunny Ohia, filed Mar. 19, 2004. This application is herein incorporated by reference in its entirety.

### BACKGROUND OF THE INVENTION

(−)-hydroxycitric acid (HCA) is the main active ingredient of the herbal extract of the dried fruit of South Asian trees of the genus Garcinia cambogia. This compound has been suggested to have antiobesity and appetite suppressive effects. See U.S. Pat. Nos. 5,783,603 and 6,638,542. Studies have also shown HCA inhibits the actions of citrate cleavage enzyme, suppress fatty acid synthesis, increase hepatic glycogen synthesis, suppress food intake, increase energy expenditure, curb appetite, reduce plasmatic cholesterol levels and inhibit fat synthesis.

Other studies have investigated the effect of hydroxycitric acid on serotonin or 5-hydroxytryptamine (5-HT) release from isolated rat brain cortex. Such studies suggest that HCA altered the baseline of spontaneous tritium efflux but had no significant effect on potassium-evoked release of 5-HT. When applied on its own, HCA elicited a concentration-dependent increase in efflux of 5-HT. Ohia, et al., Res. Commun. Mol. Pathol. Pharmacol. 2001 March-April; (3-4): 210-216.

It is known that levels of certain neurotransmitters in the brain correlate with function and/or symptoms of disease. Thus patients suffering from Parkinson's disease are known to have lowered dopamine levels and patients suffering from depression are known to have decreased levels of serotonin.

### SUMMARY OF THE INVENTION

The present invention relates to the discovery that administration of HCA increases brain cortex levels of the neurotransmitter dopamine and its metabolites 3,-4-dihydroxyphenylacetic acid (DOPAC) and homovanillic acid (HVA). Described herein are compositions and methods for increasing brain cortex levels of dopamine and/or its metabolites in a subject. Furthermore, in certain aspects of the invention, HCA is administered to a subject with sub-normal levels of dopamine or in need of enhanced dopamine levels to produce a therapeutic or prophylactic effect.

The invention provides a method of increasing dopamine levels in a subject in need thereof, comprising the step of administering an effective amount of hydroxycitric acid (HCA) to said subject, wherein the amount of HCA administered is effective to increase the dopamine levels in the subject.

In one aspect, the amount of HCA administered is effective to increase cardiovascular output in the subject.

In another aspect, the amount of HCA administered is effective to increase cognitive skills or memory retention in the subject.

In another aspect, the amount of HCA administered is effective to increase adenosine triphosphate (ATP) production in the subject.

In another aspect, the amount of HCA administered is effective to alleviate one or more complication and or symptoms associated with a condition selected from the group consisting of Parkinson's disease, Alzheimer's disease, attention deficit disorder (ADD), attention deficit/hyperactivity disorder (ADHD), obsessive/compulsive disorders, depression, bipolar disorder, schizophrenia, and addiction.

In certain aspects of the invention, the amount of HCA administered is effective to increase brain cortex dopamine levels.

The invention further provides a method of treating a disease or disorder associated with subnormal dopamine level in the brain cortex comprising the step of administering to a subject in need thereof an amount of HCA effective to increase the brain cortex dopamine and/or serotonin level in the subject.

### DETAILED DESCRIPTION OF THE INVENTION

The invention provides methods of enhancing neurotransmitter levels in a subject by administering an amount of HCA effective to increase dopamine and/or serotonin levels, or levels of metabolites, in a subject in need thereof. These increases in neurotransmitter levels can be associated with increases in health and well being of patients.

As used herein, HCA refers to hydroxycitric acid, its salts, metabolites or mixtures thereof. Preferably, calcium, magnesium, sodium or potassium hydroxycitrate or mixtures thereof are used. In particularly preferred embodiments, a double salt of HCA comprising potassium and calcium is used, e.g., CITRIMAX® or SUPER-CITRIMAX® (InterHealth Nutraceuticals, Inc, Benicia, Calif.). Additionally, single, double and triple salts of HCA comprise elements of groups I or II of the periodic table.

HCA is administered at such a dosage, in a number of dosages, and over a time period effective to increase dopamine levels in the subject. Typically, HCA is used in a daily dose of between 2 mg and 250 mg per kg body weight. In an alternative embodiment of the invention a daily dose between 4 mg and 150 mg per kg body weight. In an alternative embodiment of the invention a daily dose between 10 mg and 90 mg per kg body weight. For a human subject the quantity of HCA per daily dose would thus typically be between 100 mg and 20 grams. In an alternative embodiment of the invention the quantity of HCA per daily dose would be between 250 mg and 10 grams. In an alternative embodiment of the invention the quantity of HCA per daily dose would be between 400 mg and 6 grams. In an alternative embodiment of the invention the quantity of HCA per day would be between 500 mg and 5 grams per dose.

Whenever the term “dose” or “dosage” is used within this disclosure, any dosage form is encompassed. When administered orally, the dosage may take the form of a pill, tablet, capsule, powder, liquid composition, or admixed in food or a beverage. When administered orally, the HCA may be administered as a percentage by weight of the diet. In preferred embodiments, HCA comprises 0.05% to 5.0% of the subject's diet. In an alternative embodiment HCA comprises 0.2% to 5.0% of the subjects diet.

In certain aspects, the present invention provides therapeutic or prophylactic methods of treating one or more conditions or disorders associated with sub-normal or decreased dopamine levels. HCA can be administered to a subject in need of an increase in dopamine levels. The dopamine-enhancing amount of HCA can be used either alone or in combination with one or more other substances contributing to increasing dopamine levels in a subject (e.g., chromium, extract from kava, dopamine, a dopamine agonist, or a dopamine precursor, such as L-DOPA) and/or a substance known to alleviate one or more symptoms of the condition or disorder. Other substances that may be administered include those such as extract from green tea, extract from gymnema, or extract from ginseng.

In certain embodiments, HCA and other substances are administered to produce a synergistic effect. In such embodiments, the amount of HCA or second substance administered can be less than when either substance is administered alone and still produce the desired effect.

HCA administration can be used therapeutically for treatment of conditions characterized by depressed brain cortex levels of dopamine or wherein elevation of brain dopamine levels is useful to achieve a therapeutic effect. Such conditions include, but are not limited to, conditions and or symptoms associated with decreased cardiovascular output, e.g., congestive heart disease, Parkinson's disease, attention deficit disorder (ADD), attention deficit/hyperactivity disorder (ADHD), obsessive/compulsive disorders, depression, bipolar disorder, schizophrenia, and addictions or cravings for, e.g., sugars, nicotine, carbohydrates, alcohol, cocaine, or amphetamines.

Furthermore, by increasing cognitive skills and memory retention, HCA can be administered to decrease or slow the mental effects of aging. Moreover, HCA can be administered to regulate energy production (increase ATP production) in a subject.

### EXAMPLE

This Example demonstrates the effects of an HCA extract and fluoxetine on rat brain cortex neurotransmitters. 10 mg, 100 mg, or 250 mg, per day of HCA (corresponding with 0.2, 2 and 5% of the diet, respectively) or 15 mg/kg per day of fluoxetine were administered orally. The 5% HCA dose corresponds to 25-times the recommended dose of HCA. Animals were euthanized after 30, 60 and 90 days. Brain cortices were analyzed for serotonin (5-HT), its metabolite 5-hydroxyindoleacetic acid (HIAA), dopamine (DA), and its metabolites 3, 4-dihydroxyphenylacetic acid (DOPAC) and homovanillic acid (HVA) content utilizing reverse phase HPLC with electrochemical detection.

HCA (0.2, 2, and 5%) increased cortex 5-HT, by 11, 9, and 12%, respectively, after 90 days (p<0.05). In contrast, fluoxetine treatment decreased cortex 5-HT by 9, 7 and 8% on days 30, 60 and 90, respectively (p<0.05). Fluoxetine also decreased cortex HIAA by 19, 15 and 17% (p<0.05) 30, 60 and 90 days post-treatment, respectively. No changes in HIAA were observed in any of the HCA treatment groups.

After 90 days, HCA (0.02, 2 and 5%) increased cortex DA by 10, 15 and 18% (p<0.05) and increased DOPAC by 23, 26 and 29% (p<0.05), respectively. In contrast, fluoxetine decreased DA by 18, 15 and 19% (p<0.05), respectively. No changes in cortex DOPAC were observed in all fluoxetine groups. On day 90, HCA (0.02, 2 and 5%) increased cortex HVA by 12, 15 and 13% (p<0.05), respectively. Fluoxetine decreased cortex HVA by 17, 13 and 14% (p<0.05), respectively.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are apparent to those skilled in the art, are intended to be within the scope of the invention.

1. A method for treating a symptom in a mammal by increasing one or more neurotransmitter levels comprising:
(a) identifying a person suffering from or at risk for suffering from the symptoms; and
(b) administering a composition comprising an effective amount of (−)-hydroxycitric acid sufficient to increase levels of dopamine or serotonin in the mammal's brain.

2. The method of claim 1, wherein step (b) involves daily administering of the composition comprising an amount of (−)-hydroxycitric acid between:
about 2 mg per kg body weight of said person; and
about 250 mg per kg body weight of said person.

3. The method of claim 1, wherein step (b) involves daily administering of the composition comprising a total amount of (−)-hydroxycitric acid between:
about 100 mg; and
about 20 grams.

4. The method of claim 1, wherein step (b) involves administration of a single, double or triple salt of (−)-hydroxycitric acid.

5. The method of claim 1, wherein the symptom identified in step (a) is for Parkinson's disease or Alzheimer's disease.

6. The method of claim 1, wherein the symptom identified in step (a) is for attention deficit disorder.

7. The method of claim 1, wherein the symptom identified in step (a) is for attention deficit/hyperactivity disorder.

8. The method of claim 1, wherein the symptom identified in step (a) is for obsessive/compulsive disorders.

9. The method of claim 1, wherein the symptom identified in step (a) is for depression.

10. The method of claim 1, wherein the symptom identified in step (a) is for bipolar disorders.

11. The method of claim 1, wherein the symptom identified in step (a) is for schizophrenia.

12. The method of claim 1, wherein the symptom identified in step (a) is dysfunctional cognitive skills.

13. The method of claim 1, wherein the symptom identified in step (a) is for dysfunctional energy regulation.

14. The method of claim 1, wherein the symptom identified in step (a) is for dysfunction due to aging.

15. The method of claim 1, wherein the symptom identified in step (a) is selected from the group consisting of cravings for sugars, carbohydrates, alcohol, nicotine, cocaine, and amphetamine.

16. The method of claim 1, wherein the symptom identified in step (a) is selected from the group consisting of addiction to nicotine, alcohol, cocaine, and amphetamine.

17. The method of claim 1, wherein step (b) involves administering the composition comprising one or more additional compounds selected from the group consisting of chromium, extract from kava, dopamine, a dopamine agonist, a dopamine precursor, gymnemic acid extract, green tea extract, and ginseng extract.

18. A method for reducing adverse symptom in a person having a dopamine or serotonin deficiency comprising:
(a) assaying for one or more neurotransmitters or their metabolites selected from the group of dopamine, 3,-4-dihydroxyphenylacetic acid, homovanillic acid, 5-hydroxyindoleacetic acid and serotonin; and
(b) administering a composition comprising an effective amount of (−)-hydroxycitric acid.

19. The method of claim 18, wherein step (b) involves daily administering the composition comprising an amount of (−)-hydroxycitric acid between:
about 2 mg per kg body weight of said person; and
about 250 mg per kg body weight of said person.

20. The method of claim 18, wherein step (b) involves daily administering the composition comprising a total amount of (−)-hydroxycitric acid between:
about 100 mg; and
about 20 grams.

21. The method of claim 18, wherein the neurotransmitters or their metabolites identified in step (a) are selected together with indications of disorders selected from the group of Parkinson's, ADD, ADHD, obsessive/compulsive disorders, depression, bipolar disorders, schizophrenia, dysfunctional cognitive skills, dysfunctional energy regulation, and dysfunctions due to aging.

22. The method of claim 18, wherein the neurotransmitters or their metabolites identified in step (a) are selected together with symptom selected from the group consisting of addiction to alcohol, nicotine, cocaine, and amphetamine.

23. The method of claim 18, wherein the neurotransmitters or their metabolites identified in step (a) are selected together with symptom selected from the group consisting of cravings for sugars, carbohydrates, alcohol, nicotine, cocaine, and amphetamine.

24. The method of claim 18, wherein step (b) involves administering the composition comprising one or more additional compounds selected from the group consisting of chromium, extract from kava, dopamine, a dopamine agonist, a dopamine precursor, gymnemic acid extract, green tea extract, and ginseng extract.

25. The method of claim 18, wherein step (b) involves administration of a single, double or triple salt of (−)-hydroxycitric acid.
